(19) United States
(12) Patent Application Publication
(10) Pub. No.: US 2005/0205093 A1
(43) Pub. Date: Sep. 22, 2005

(54) Method and program for calulating ventilator weaning duration

(76) Inventor: Ernest R. Jabour, Las Cruces, NM (US)

Correspondence Address: Edward F. O'Connor, Suite 150, 1920 Main Street, Irvine, CA, 92614, US

(21) Appl. No.: 11/078,159

(22) Filed: Mar. 11, 2005

Related U.S. Application Data
(60) Provisional application No. 60/554,091, filed on Mar. 16, 2004.

(51) Int. Cl. A62B 7/00; A61M 16/00

(52) U.S. Cl. 128/204.23

(57) ABSTRACT

A method, program and computer system for a method for calculating proper treatment for weaning a patient from respiratory ventilator, based on the respiratoryvital signs of that patient.

The present application claims priority under 35 USC §119(e) from U.S. provisional application Ser. No. 60/554,091, filed 16 Mar. 2004 and entitled a “Method and Program for Calculating Ventilator Weaning Duration.”.

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates generally to medical 15 pulmonary function tests and more particularly to a method and software program for spirometry diagnosis to predict when a patient may be removed from a mechanical ventilator to breath on his own.

2. Background of the Invention

A patient may need to be placed on an assisted ventilation device, a ventilator, as a result of injury, surgery, disease or adverse drug reactions. In these cases the ventilator is used for a determinate amount 25 of time until a patient can again breath on their own. It is beneficial to wean a patient from a ventilator as their lungs and diaphragm become more functional, allowing there own muscles and lungs to undertake the task of respiration. Predicting the ability of a 30 patient to breathe independently after being removed from mechanical ventilation, is and has been an art and a science. Weaning from mechanical ventilation can be defined as the process of abruptly or gradually withdrawing ventilatory support. Some patients will need to be progressively withdrawn from mechanical ventilation, which requires that the patient's respiratory condition and ability to be withdrawn be estimated. The use of respiratory parameters such as tidal volume, vital capacity, respiratory rate, and maximum inspiratory force, to predict weaning success has been inaccurate for this purpose.

It is generally not necessary to predict when a patient need be removed from a mechanical ventilator in a straightforward cases, such as post-operative recovery, where there is no preoperative lung disease. In the case of patient who is intubated and placed on a mechanical ventilator for respiratory failure for, for example, pneumonia or congestive heart failure, the ability to predict when a person is ready to come off is not as easy. This is because the parameters used to measure readiness to wean a patient from a ventilator 20 alone are not particularly accurate by themselves.

Weaning from mechanical ventilation is usually started only after the underlying disease process that necessitated mechanical ventilation has significantly improved or is resolved. Leaving a patient on a mechanical ventilator for too long a time, however, may cause injury to the patient, such as contracting pneumonia, barotrauma or muscle atrophy.

Respiratory muscles do work for a given breath when they contract with a given pressure Psb, for a given time Ti, where Ti is the inspiratory breath time. Methods exist for predicting respiratory muscle fatigue, however many are impractical. For example, muscle fatigue has been shown to be occurring when:
Pdi/Pdimax×Ti/Ttot>0.15

where Pdi is diaphragmatic inspiratory pressure, Pdimax is the maximum diaphragmatic pressure and Ttot is the total breath cycle time including both inspiration and expiration. The measurements of Pdi and Pdimax however, require an esophageal balloon catheter and pressure transducer and thus are not practical. A weaning index based on respiratory mechanics, respiratory muscle function, and gas exchange has been proposed in the past, for example Jabour, E R, Rabil, D M, Truwit, D, et al (1991) Evaluation of a new weaning index based on ventilatory endurance and efficiency of gas exchange. Am Rev Respir Dis 144, 531-537.
WI=PTI×(Ve 7.4/Vtsb), (PTI a pressure-time index) 20
WHEREAS THE WI FOR THIS ONE IS
WI=Psb/NIF×0.4×Ve 7.4/Vtsb

It would therefore be advantageous to have an improved method and that can be readily implemented in a computer program to monitor a patient's respiratory strength. It would further be advantageous to likewise have a method that can be readily implemented by a computer program to evaluate a patient's strength and stiffness, so as to enable the physician to evaluate evolving lung problems and indicate the appropriate course of treatment.

### SUMMARY OF THE INVENTION

A solution to the above has been devised. The present invention provides a technique method, computer program product and system to calculate the ability of patients who are on a mechanical ventilator and predict when they may be best removed from ventilator assistance. A series of vital sign parameters are used to calculate the correct time for weaning that is better and more accurate at predicting optimal weaning time. Use of the equation reduces ventilator time, helps to track the progress of a patient, and allows communication about the patient progress between health care providers.

In order to more accurately predict outcome of removal of the patient from ventilatory support, weaning parameters are combined into a equation resulting in a weaning index. Numerical weaning parameters are combined in a way that their position in the weaning index equation (their place in the numerator or the denominator) are determined by whether a given particular parameter is either directly or indirectly proportional to a successful weaning outcome, with respect to a given change in direction of values for all the parameters.

Respiratory muscle fatigue can be implied from simple bedside measurement of respiration, the Vtmv, Ppeak, PEEP, and Vtsb measurements to calculate a weaning index. This weaning index may be used to reliably predict whether a patient is a candidate for removing assisted mechanical ventilation. The index may be implemented in a computer program for ready use by the physician, or other health care provider, and with reliance on a flow sheet over time. This method may be automated as a computer program that logs patient parameters to produce a flow sheet of patient progress.

The calculation may be written as a program and loaded on a microprocessor-based digital computer, such as a personal computer or a Personal Digital Assistant (PDA) handset, which allows availability and ease of calculation of the index of weaning. The PDA program automatically calculates the variables into the equation and makes the calculation. The data may be entered by, for example, a respiratory therapist in the ICU or other ventilator care unit may chart a patient's progress the program product will automatically chart and predict the weaning readiness, automatically calculated from the entries that are charted. The use of this method and program also allows a reduction in ventilator days and earlier triage of patients to longer term ventilator units if they were not making progress. A bedside measurement of Ve, RR, with a calculated Vtsb, a calculated Psb derived from the Ppeak-PEEP and Vtmv found on a ventilator flow sheet, as well as the Ve obtained during blood gas measurements and adjusted for Ve7.4 or Vnat, will allow accurate prediction of weaning outcome. It also allows a planned approach to troubleshooting problem areas and lends itself well to protocols used for weaning. Other features and advantages of the present invention should be apparent from the following description of the preferred embodiment, which illustrates by way of example, the principles of the invention.

### BRIEF DESCRIPTION OF THE DRAWINGS

FIG. 1 is a flow diagram of a method of the present invention;

FIG. 2 is a flow chart of use of the program product of the present invention;

FIG. 3 is a flow chart of an embodiment of the sequence of operations performed by the program product and computer as a system;

FIG. 4 is a block diagram of an exemplary computer that may be used in conjunction with the program product of the present invention; and

FIGS. 5A and 5B are screenshots of a preferred embodiment of a computer program of the present invention.

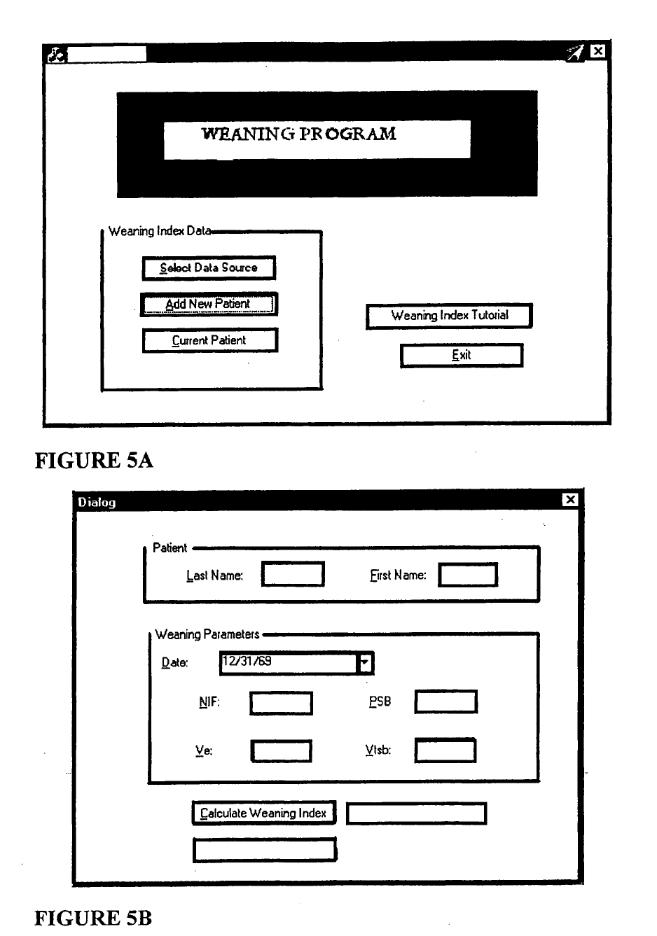

### DESCRIPTION OF THE INVENTION

The following description, and the figures to which it refers, are provided for the purpose of describing examples and specific embodiments of the invention only and are not intended to exhaustively describe all possible examples and embodiments of the invention.

Various terms are used herein to refer to aspects of the present invention. To aid in the clarification of the description of the components of this invention, the following definitions are provided. Absent specific reference otherwise, these definitions are neither meant to be limiting nor construed as otherwise ordinarily known and understood in the art.

Arterial Blood Gas analysis (ABG)—a lab test of arterial blood which measures carbon dioxide and oxygen levels as well as acid-base status. A normal ABG may return values of a partial pressure of oxygen (PAO2) of 75 to 100 mm Hg; partial pressure of carbon dioxide (PACO2) of 35 to 45 mm Hg; a pH of 7.35 to 7.45; an oxygen saturation (SaO2) of 94% to 100% and a bicarbonate (HCO3) of 22 to 26 mEq/liter; Diaphragmatic inspiratory pressure (Pdi)—the pressure created by the diaphragm and Pdimax is the 10 maximum diaphragmatic pressure;

Inspiratory breath time (Ti)—Ttot is the total breath cycle time including both inspiration and expiration;

Minute ventilation (Ve)—the volume of air breathed per minute, Ve7.4 or minute ventilation adjusted to give a normal pH of 7.4;

Negative inspiratory force or pressure (NIF or NIP)—the maximal inspiratory force generated with the airway occluded. Reflects inspiratory muscle strength;

Peak airway pressure (Ppeak)—recorded on a ventilator flow sheet for a given ventilator delivered tidal minute volume Vtmv;

Positive End Expiratory Pressure (PEEP)—a method of mechanical ventilation in which pressure is maintained to maintain a set airway pressure at the end of an expiratory phase of mechanical ventilation, thus keeping alveoli open and improving gas exchange;

Spontaneous tidal volume (Vtsb)—the volume of a normal inspiration or expiration during relaxed breathing;

Spontaneous breath pressure (Psb)—the pressure required by the patient to take a spontaneous breath (Vtsb) which is measured when weaning;

Personal digital assistant (PDA)—a small handheld microprocessor-based computer;

Respiratory rate (RR)—number of breaths/minute;

Tidal volume (Vt)—volume of a normal inspiration or expiration during relaxed breathing, mechanical ventilator set tidal volume (Vtmv) is the tidal volume of mechanical ventilator which is set by the operator.

Generally, there are five main parameters used in conjunction with ventilators for respiratory support of patients who cannot breathe on their own: tidal volume (Vtmv), respiratory rate RR, FIO2 (Fraction of Inspired Oxygen), PEEP and the I:E ratio (time for inspiration in relation to time for expiration). The weaning index of the present invention may be calculated, for example, by taking physiological respiratory values of the following. Their relative position in the weaning index equation is determined by whether an increase in value of a given particular parameter is either directly or indirectly proportional to weaning outcome: minute ventilation (Ve), (normally 5-8 liters per minute); spontaneous tidal volume (Vtsb); negative inspiratory force (NIF) and spontaneous breath pressure (Psb), the pressure required by the patient to take a spontaneous breath, which is measured when the weaning parameters are obtained.

According to the method of the present invention if an increase in value of the weaning parameter favors a successful weaning outcome, such as Vtsb, then the parameter is placed in the denominator of the weaning equation. The increase in Vtsb is directly proportional to weaning success and thus goes in the denominator. Conversely, a parameter whose increasing value does not favor weaning success, for example minute ventilation Ve, would go in the numerator. Increases in Ve are therefore inversely proportional to weaning success. In accord with this general, principal increases in the NIF are in the denominator and increases in Psb are placed in the numerator and so on.

The spontaneous breath pressure is calculated by the equation:
Psb=(Ppeak−PEEP)×Vtsb/Vtmv
where Ppeak is the peak airway pressure recorded on a ventilator flow sheet for a given ventilator delivered tidal volume Vtmv. The Psb for a given Vtsb is proportional to the peak airway pressure from which PEEP must be subtracted in the same proportion of the Vtsb to the Vtmv. Vtsb is calculated by dividing the spontaneous Ve by the spontaneous respiratory rate RR, Ve is divided by respiratory rate to give the average Vtsb.

Psb can be calculated from data found at the bedside from the flow sheet and respiratory muscle fatigue has been shown when spontaneous breath pressure is greater than 40% of the maximal inspiratory force (Psb/NIF>0.4) or when Psb/NIF×Ti/Ttot is greater than 0.15. There is a large overlap in values for Psb/NIF and Psb/NIF×25 Ti/Ttot between successfully weaned and unsuccessfully weaned groups of patients.

The parameters along with Psb and Ti/Ttot may be arranged in a equation in accordance with the proportionality relationships described above to predict a weaning index WI, weaning outcome as follows:
WI=Psb/NIF×Ti/Ttot×Ve7.4/Vtsb (1)

Ve effectively represents the minute ventilation at the time an arterial blood gas measurement is obtained on the day of weaning. This value of Ve is adjusted to give a normal pH of 7.40, as a measurement of the ventilation status of a patient.

$$Ve 7.4 = \frac{Ve X {Pa CO}_{2} + [ ( 7.4 - pH ) \times 125 ]}{{PaW}_{2}} \qquad ( 2 )$$

where V7.40 is a minute ventilation adjusted to give a normal pH. The equation (2) can be restated to reflect this change in Ve as:
WI=Psb/NIF×Ti/Ttot×V7.40/Vtsb (3)

A value for WI>3.5 in (4) is associated with a greater than 95% chance of weaning failure and a WI of less than 3.5 with a greater than 95% chance of weaning success.

Measurement of Ti/Ttot directly however is cumbersome and does not increase accuracy to the equation. This value in previous studies has been seen to be 0.4 in spite of the weaning outcome. Thus a new equation with a value of 0.4 for Ti/Ttot is:
WI=Psb/NIF×0.4×Ve7.4/Vtsb (4)

where Ve7.4 a patient's minute ventilation and adjusted to give a normal pH takes into account chronic stable resting elevations in PACO2. If the value of 0.4 is eliminated from the equation altogether, then WI of 3.25 would change accordingly to 3.25/0.4=8.125. The new value for WI by eliminating the value of 0.4 from the equation is:
WI=Psb/NIF×Ve7.4/Vtsb (5) or

For a WI of less than 8.125 calculated by (5), weaning success would be predicted and for a WI of greater than 8.75 and weaning failure would be expected. If equation (4) is used, a WI less than 3.25 predicts successful weaning and WI of greater than 3.5 predicts weaning failure.

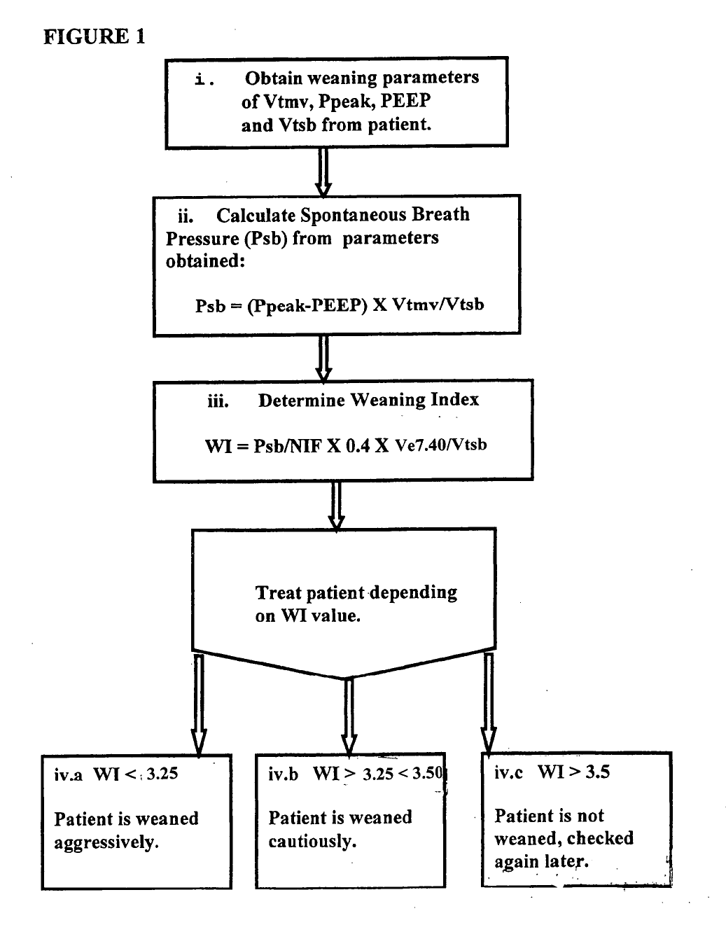

Referring now to FIG. 1, the preferred method for calculating the weaning index WI of the present invention includes the steps of:
(i) Obtaining weaning parameters of Vtmv, Ppeak, PEEP, and Vtsb and ABGs from a patient.

This is preferably done after withholding sedation, with the patient fully awake. The spontaneous minute ventilation (Vsp) and spontaneous respiratory rate (RRsp) of the patient are taken. The tidal volume Vtsb should be obtained as the Vesp/RRsp to give an average Vtsb. Vemv should be obtained from the ventilator flow sheet at the time arterial blood gases were obtained and a calculation of the desired Ve to achieve either a normal pH Ve7.40. This should be calculated by the equation:

$$Ve 7.4 = \frac{Ve X {Pa CO}_{2} + [ ( 7.4 - pH ) \times 125 ]}{{PaW}_{2}}$$

Ve is the minute ventilation obtained during ABGs and PACO2 (on the ventilator) here is from the ABGs.
(ii) Psb is calculated from obtained values from the equation:
   Psb=(Ppeak−PEEP)×Vtmv/Vtsb
   where Ppeak is the peak airway pressure during the mechanical ventilator delivered breath Vtmv and Vtsb is the spontaneous tidal volume.
(iii) The weaning index is determine by the equation 5
   WI=Psb/NIF×0.4×V7.40/Vtsb

A course of treatment is then followed according to the resulting weaning index.
(iv.a) if the value for WI<3.25 the patient is weaned aggressively. If greater than 3.25 weaning should be delayed and a search for how parameters such as PPeak and NIF can be improved should be conducted.

The IMV (intermittent mandatory ventilation) is reduced to 4. A little pressure support of 3-5 cm H20 may be added to overcome the resistance of the ventilator circuit and tubing. Arterial blood gasses should be obtained to determine stability of the patient about 30 minutes after the ventilator setting is changed. If the vital signs are okay then the patient may be placed on CPAP of 5 cm H20 with no pressure support at ABGx should be repeated and if the calculated Ve7.40 increases, this value should be used in recalculating a weaning index. extubated.
(iv.b) if WI is >3.25 but <3.5 then a more cautious weaning is indicated.

In this setting decrease in IMV are done more gradually, for example IMV to 8 with ABGs in one half hour and the reduce IMV by 2 every two hours, with ABG checks one half hour after each change. The weaning process may proceed as long as the patient can tolerate it. Also evaluate the variables contained in the equation for weaning index to see if any of them can be altered to improve the results as outlined above. Ve requirements may increase due to pulmonary edema or poor lung compliance with high Ppeak due to pulmonary infiltrates, or pleural or chest wall processes.
(iv.c) if WI>3.5, an attempt is made to change variables for a better outcome but no is made to wean.

Interventions to change the parameters, medical treatment to improve respiratory efficiency, can include nutritional support and a respiratory strengthening regimen of periods of the patient assuming some of the work of breathing alternating with periods of rest.

Care should be taken in measuring NIF. In practice the measurement of NIF is subject to great variability and thus a protocol for their measurement is advised to assume the greatest accuracy of patient effort possible. This includes eliminating endotracheal tube cuff leaks assuring maximum cooperation of patients and using 20 second breath holding techniques outlined by marini which allows a patient to exhale through the endotracheal tube but inhalation is occluded in a system connected to a pressure transducer for a period of 20 seconds. Also efforts to measure the work of breathing which is derived from airway pressures of the mechanically ventilated patient's bedside ventilator flow sheet, are the most accurate when the patient is resting and not making respiratory efforts which tend to underestimate the distending pressure of the lungs and chest wall. This is most accurate in the assist control made with the patient's entire minute ventilatory requirements being met. The peak airway pressure is measured and recorded on the ventilator flow sheet and, again, is an accurate indication of the true distention pressure of the lungs and chest wall when the patient is at rest, preferably asleep and with the ventilator on assist control mode.

From the peak airway pressure Ppk the value PEEP must be subtracted to derive a true distending pressure for the ventilator delivered tidal volume. From this pressure the pressure required to take a spontaneous breath (Psb) can be calculated. If patients make respiratory efforts during mechanical ventilator delivered breaths, the peak airway pressures underestimate the true distending pressure and thus the calculated spontaneous breath pressures are underestimated.

Once it is determined that a patient is ready to wean by an index of weaning less than 3.25, by equation (4), it is unnecessary to take much time to achieve this end. The ventilator rate is reduced to 4 on IMV mode and 10 arterial blood gas is obtained half an hour thereafter. If the patient does well during this time and the Ve does not increase significantly, they are placed on CPAP of Scm H20 and a new ABG is obtained in a half hour and if the Ve doe not increase significantly the patient is extubated.

If the weaning index is greater than 3.25, this indicates which factors in the equation might be altered to reduce values for WI, such as a greater NIF or a lower Ve. Interventions may also be implemented to improve respiratory muscle strength and/or lower minute ventilation. Respiratory muscle strength may be worsened through poor nutrition and resulting electrolyte abnormalities. Normalizing these may improve respiratory muscle strength. Periods after the use of paralytic agents may also be followed by generalized muscle weakness, so withholding narcotic analgesics may also improve respiratory muscle strength.

After correcting the above factors, respiratory muscle training to improve respiratory muscle strength may be achieved by letting the patient assume intervals of reduced mechanical ventilatory support followed by periods of rest. Improvement in minute ventilatory requirements may be improved by identifying and treating causes for increased ventiltory demands. Pulmonary edema thus treated by diuresis and pneumonia treated by antibiotics can correct higher ventilatory requirements.

Improvement in lung compliance will reduce Psb. Factors that worsen lung compliance by increasing the elastance of the respiratory system, for example pleural effusions, pulmonary edema, abdominal distention, or pulmonary infiltrates may be identified and corrected to reduce Psb. On the other hand compliance or the lung may be decreased by increases in airway resistance pressures as seen in COPD or asthma. Reductions in airway resistance may be affected by bronchodilators or anti-inflammatory medications to reduce airway inflammation.

If there are no correctable causes of an elevated weaning index then patients ought to receive full ventiltory support with attempts at training respiratory muscles, if this seems appropriate, by allowing brief periods of less supported ventilation by reducing ventilator rates. After this period of therapeutic intervention, weaning assessment may be repeated. An examination of the equation reveals that it is a balance between supply of respiratory muscle strength with its attendant endurance and respiratory demand as minute ventilatory requirements supplied by spontaneous tidal volumes to get a required respiratory frequency.

The weaning index may be elevated due to erroneous collection of parameters. The measurement of NIF is subject to operator error, so it preferred that a rigid protocol for collection of parameters be used, a protocol sufficient to measure maximum inspiratory effort even in an uncooperative patient.

One method, know to those of skill in the art, allows expiration through the ET tube and occludes the ET tube during inspiration for a period of twenty seconds. A patient, even if uncooperative, will usually give their maximum effort and this maneuver also allows the patient to blow down to RV, a point of maximum diaphragmatic strength. A device with a one-way valve and a pressure manometer can be devised for this purpose. This method was described by Marini.

A bedside measurement of Vesp, RRsp, with a calculated Vtsb, a calculated Psb derived from the Ppeak−PEEP and Vtmv found on a ventilator flow sheet, as well as the Ve obtained during blood gas measurements and adjusted for V7.4 will be useful in prediction of weaning outcome. It also allows a planned approach to troubleshooting problem areas and lends itself well to protocols used for weaning.

The method of the present invention can be implemented on a microprocessor-based computer system as well.

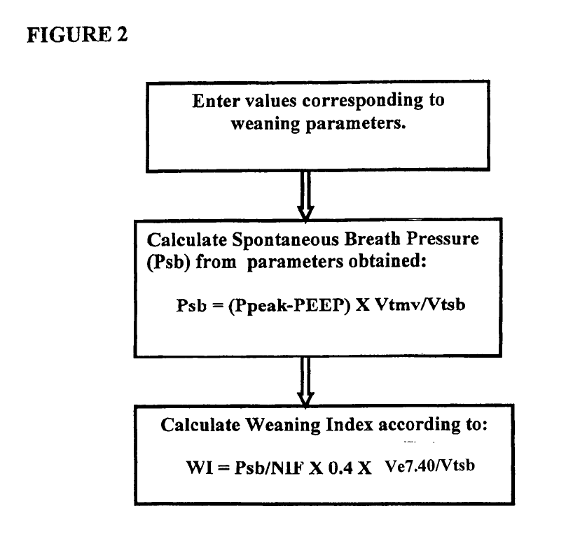

FIG. 2 is a flow chart for any program product of the present invention with a computer or computer system. The weaning parameters, for example, of negative inspiratory force, arterial blood gases, spontaneous minute volume (Vesb) and spontaneous rate of respiration (RRsb) are determined. Spontaneous tidal volume Vtsb is calculated from these values as Vesp/RRsp. Then from this value spontaneous breath pressure Psb is calculated as ((Ppk−PEEP)×Vtsb/Vtmv). The weaning index of the present invention may then be calculated as WI=Psb/NIF×0.4×Ve7.4/Vtsb.

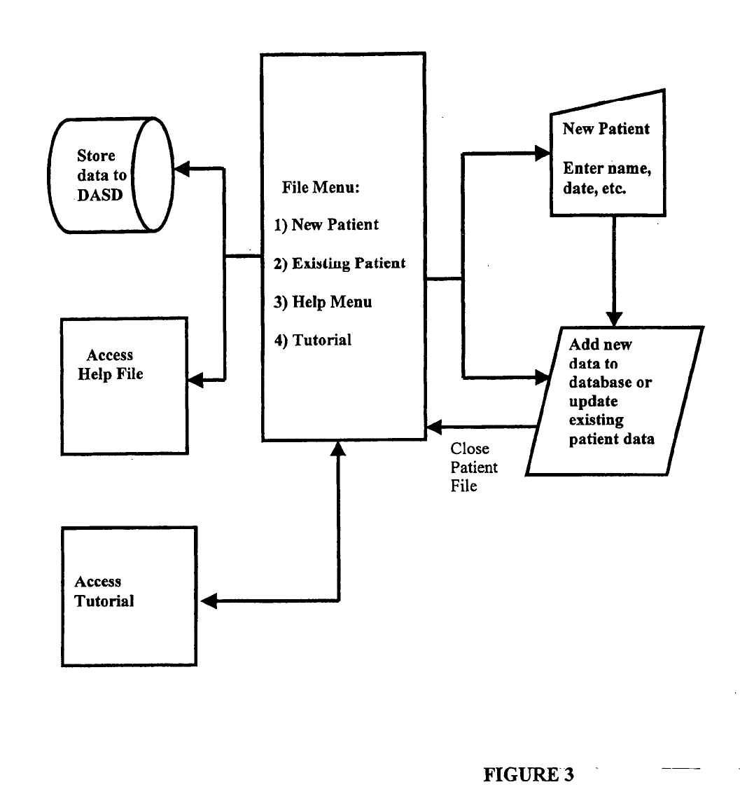

FIG. 3 is a flow chart of the preferred embodiment of the sequence of operations performed by the program product and computer as a system. The user initially selects an option to enter data on a new or existing patient from a screen. This and other screens made be programmed by methods known to those of skill in the art, for example in hypertext-markup (html) language and with http protocol if used on the Internet global communications network. Optimally a Help and Tutorial options are also provided, directing the user to view Help or Tutorial pages or files accordingly. If a new patient is selected a new database file is opened. If an existing patient name is selected there is, by definition, an existing database file, which is opened. The Weaning Index is calculated and the data is saved to the database file. The data may then be stored for future reference.

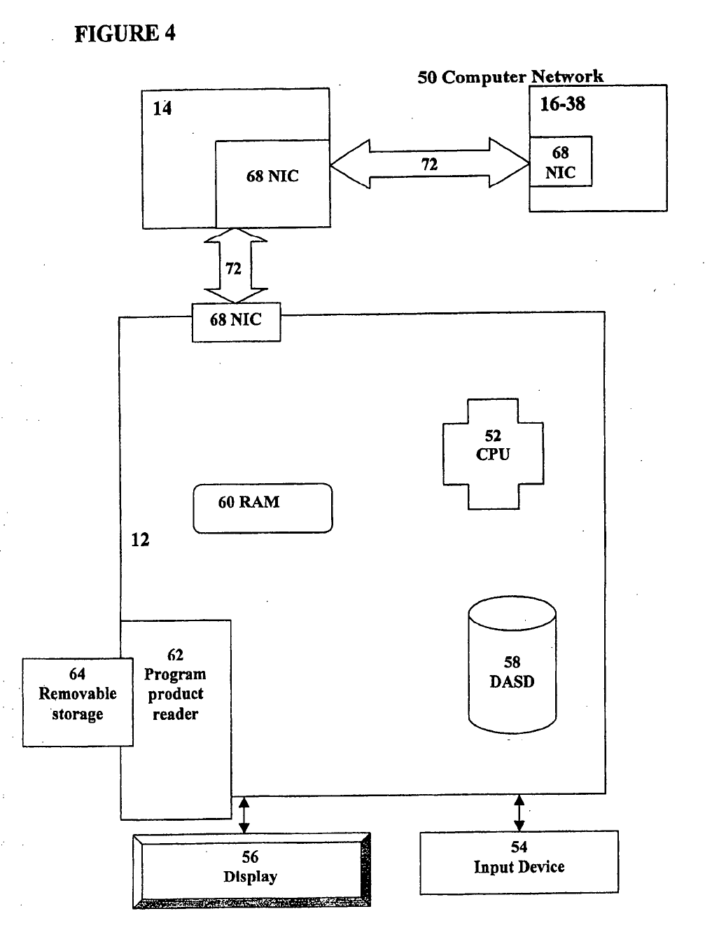

FIG. 4 shows exemplary computers, 14, 16-38 and (shown in more detail) 12 of the type that might comprise a network system of two or more users 12, such as the Internet global communications network, that might be used with the computer program of FIG. 3, either individually on a personal computer or Personal Digital Assistant, or over a network. It is understood that a personal computer, Web TV, kiosk Personal Digital 30 Assistant or equivalent terminal device for accessing a computer network may be used to access the network.

Each computer operates under control of a central processor unit (CPU) 52, such as a “Pentium” microprocessor and associated integrated circuit chips, available from Intel Corporation of Santa Clara, Calif., USA. A computer user can input commands and data from a keyboard, computer mouse or equivalent device 54, and can view inputs and computer output at a display 56. The computer 12 also includes a direct access storage device (DASD) 58, such as a hard disk drive, floppy disk or other recordable media. The memory 60 typically comprises volatile semiconductor random access memory (RAM).

The member computer 12 may preferably include a program product reader 62 that accepts a program product storage device 64, from which the program product reader can read data (and to which it can optionally write data). The program product reader can comprise, for example, a disk drive, and the program product storage device can comprise removable storage media such as a magnetic floppy disk, a CD-R disc, a CD-RW disc, or DVD disc.

Each computer shown here as 12, 14 and 16-38 can communicate with another computer over the computer network 50 (such as through the Internet, an intranet or LAN) through a network interface 68 that enables communication over a connection 72 between the network 50 and the computer. The network interface 68 typically comprises, for example, a Network Interface Card (NIC) or a modem that permits communications over a variety of networks.

The CPU 52 operates under control of programming steps that are temporarily stored in the memory 60 of a computer. When the programming steps are executed, the computer performs its functions. Thus, the programming steps implement the functionality described above. The programming steps can be received from the DASD 58, through the program product storage device 64 of a recordable media, or even through a network connection 72. The program product storage drive 62 can receive a program product 64, read programming steps recorded thereon, and transfer the programming steps into the memory 60 for execution by the CPU 52. As noted above, the program product storage device can comprise any one of multiple removable media having recorded computer readable instructions, including magnetic floppy disks and CD-ROM storage discs. Other suitable program product storage devices can include magnetic tape and semiconductor memory chips. In this way, the processing steps necessary for operation in accordance with the invention can be embodied on a program product.

Alternatively, the program steps can be received into the operating memory 60 over the network 50. In the network method, the computer receives data including 25 program steps into the memory 60 through the network interface 68 after network communication has been established over the network connection 72 by well-known methods that will be understood by those skilled in the art without further explanation. The program steps are then executed by the CPU 52 thereby comprising a computer process.

It should be understood that the above description of the construction of the computers 12-38 of a computer network are for illustrative purposes. It will be understood by those of skill in the art to not only include the above structural description but can have an alternative construction, so long as the computer acting as a server computer can communicate with the other node of the computer system, computers 16-38 for example, acting as a user computer sufficient to support the program functionality described herein.

It will be appreciated that the invention has been described hereabove with reference to certain examples or preferred embodiments as shown in the drawings. Various additions, deletions, changes and alterations may be made to the above-described embodiments and examples without departing from the intended spirit and scope of this invention. Accordingly, it is intended that all such additions, deletions, changes and alterations be included within the scope of the following claims.

1. A method for treating a patient on a ventilator by calculating the remaining time a patient is required to be on the ventilator before being weaned, comprising the steps of:
obtaining weaning parameters of Vtmv, Ppeak, PEEP, and Vtsb from a patient, calculating Psb from the parameters with the equation
Psb=(Ppeak−PEEP)×Vtmv/Vtsb
calculating a weaning index WI with the equation
WI=Psb/NIF×0.4×V7.40/Vtsb, and
administering weaning treatment to a patient according to the weaning index. 15

2. The method as defined in claim 1, wherein the weaning index is less than 3.25 and the weaning treatment comprises aggressive weaning from the ventilator.

3. The method of claim 2, wherein the aggressive weaning treatment comprises the reducing ventilator minute volume and testing the arterial blood gases of the patient about 30 minutes after the ventilator setting is changed and, if vital signs are sufficient reducing the patent to CPAP 5 cm H20 and repeating arterial blood gases in a half hour. If Ve7.40 is not significantly increased, extubating the patient.

4. The method of claim 2, wherein the aggressive weaning treatment comprises the reducing ventilator minute volume and testing the arterial blood gases of the patient about 30 minutes after the ventilator setting is changed and reducing the patient to CPAP 5 cm H20 and repeating ABGs in 30 minutes and Ve7.40 is not significantly higher intervals and, if vital signs are sufficient, extubating the patient. These calculations of Ve7.40 are automatically made with the program in the hand held computer and recalculations of WI can be done just prior to extubation using the same NIF Psb, Vtsb Vtmv as before and substituting only the last Ve7.4 from ABGs on CPAP 5 cm H20

5. The method as defined in claim 1, wherein the weaning index is greater than 3.25 and less than 3.5 and the weaning treatment comprises cautious weaning from the ventilator.

6. The method of claim 5, wherein the cautious weaning treatment comprises the reducing ventilator minute volume two or more times and testing the arterial blood gases of the patient about 30 minutes after the ventilator setting is changed each time and, if vital signs are sufficient, extubating the patient fi the last Ve7.4 on CPAP is not increased.

7. The method claim of claim 6 further including the step of administering medical treatment to improve respiratory efficiency to the patient to change the parameters used.

8. The method as defined in claim 1, wherein the weaning index is greater than 3.5 and the weaning treatment comprises medical treatment to improve respiratory efficiency and a second calculation of weaning index if the patient shows sings of improvement on a daily basis.

9. A program product for use in a computer that executes program steps recorded in a computer-readable media for treating a patient on a ventilator by calculating the remaining time a patient is required to be on the ventilator before being weaned, comprising:
a recordable media; and
a program of computer-readable instructions executable by the computer to perform a method comprising:
data entry of weaning parameters of Vtmv, Ppeak, PEEP, and Vtsb from a patient,
calculating Psb from the parameters with the equation
Psb=(Ppeak−PEEP)×Vtmv/Vtsb calculating then displaying a weaning index WI with 15 the equation
WI=Psb/NIF×0.4×Ve7.40/Vtsb.

10. A computer server system for responding to requests for network data files, the server system comprising:
a central processing unit that can establish communication with the network;
program memory that stores programming instructions that are executed by the central processing unit such that the server system establishes communication with the network and communicates with a network user, such that the server system receives a request from a network user for a network data file, and
a program that can be executed by the central processing unit responsive to a network user request that executes program steps for treating a patient on a ventilator by calculating the remaining time a patient is required to be on the ventilator before being weaned, having executable instructions to perform a method comprising:
data input of weaning parameters of Vtmv, Ppeak, PEEP, and Vtsb from a patient,
calculating Psb from the parameters with the equation
Psb=(Ppeak−PEEP)×Vtmv/Vtsb
calculating then displaying a weaning index WI with the equation 25
WI=Psb/NIF×0.4×Ve7.40/Vtsb.

11. The computer server system of claim 10 where the network data files comprise web site pages and the network is the Internet global communications network.

12. The computer server system of claim 11 wherein the server system is adapted to provide content by determining content to be provided and providing a uniform resource locator (URL) from which the determined content will be obtained.

13. A server system as defined in claim 11, wherein the network request complies with the hypertext transfer protocol (http) specification.
